(19) United States
(12) Patent Application Publication
(10) Pub. No.: US 2026/0345350 A1
(43) Pub. Date: Nov. 5, 2026

(54) METHOD AND DEVICE FOR PRODUCING A PROSTHESIS FOR A BODY PART OF A LIMB

(71) Applicant: LIFELIKE PROSTHETICS GMBH, Luckenwalde (DE)

(72) Inventor: Stefan VON ESSEN, Luckenwalde (DE)

(73) Assignee: LIFELIKE PROSTHETICS GMBH, Luckenwalde (DE)

(21) Appl. No.: 19/487,300

(22) PCT Filed: May 23, 2024
(86) PCT No.: PCT/EP2024/064264

§ 371 Date: Nov. 24, 2025

(30) Foreign Application Priority Data
May 26, 2023 (EP) 23175583.6

(51) Int. Cl. A61F 2/50; A61F 2/58

(52) U.S. Cl. CPC A61F 2/5046; A61F 2/586; A61F 2002/505; A61F 2002/5053

(57) ABSTRACT

The invention relates to a method and a device (100) for manufacturing a prosthesis (10) for a body part of a limb. On the basis of the digital representation of the impaired body part (20) and of the mirrored digital representation of the intact body part, a digital representation of a prosthesis (22) for the impaired body part is created. Consequently, the prosthesis (10) does not have to be designed from scratch, but can instead be based on the shape of the intact body part due to the chirality of limbs. At least one prosthesis (10) for the impaired body part is then manufactured using the digital representation of the prosthesis (22) and a 3D printer (106) with a print resolution of less than 0.1 mm.

The invention relates to a method and a device for manufacturing a prosthesis for a body part of a limb. In humans and animals, a limb is a paired body appendage moved by muscles. Examples are fingers, hands, arms, legs, feet and the like.

Prostheses are used to replace or complete missing or diminished body parts. As is known, a rough prosthesis blank is first created which is gradually custom-adapted to the shape and form of the affected body part by an orthopedic technician. The patient often has to make frequent visits to the orthopedic technician to adjust the custom-made prosthesis. In particular when it comes to producing lifelike prostheses, fine surface structures have to be manually sculpted into the custom prosthesis. Accordingly, significant time is required to manufacture a custom prosthesis, which means that lifelike prostheses are not affordable for everyone. Moreover, if the custom-made prosthesis is damaged or lost, the complex manufacturing process has to be carried out again from scratch.

It would therefore be desirable to simplify the manufacture of lifelike prostheses.

The object underlying the invention is therefore now to provide a device and a method for producing custom prostheses in a simplified and inexpensive manner.

The object of the invention is achieved by a method and a device for manufacturing a prosthesis for a body part of a limb according to the independent claims. Preferred further developments constitute the subject matter of the respectively subordinate subclaims.

A first aspect relates to a method for manufacturing a prosthesis for a body part of a limb.

In one method step, a digital representation of an impaired body part of a limb and a digital representation of an associated intact body part of the limb are created. An impaired body part is, for example, a completely or partially missing or damaged body part. In other words, the digital representations are a digital model or copy of the physical body parts. The digital representations are preferably created as high-density meshes using a 3D scanner and/or an imaging method such as computed tomography (CT), digital volume tomography (DVT) and the like. The quality of the digital representations is preferably such that fine surface structures of the body parts are included. Fine surface structures are for example structures that are distinguishable in width and/or depth with a resolution of below 0.1 mm, for example of 0.09 mm to 0.01 mm, preferably of 0.05 mm.

In a further method step, a digital representation of a prosthesis for the impaired body part is created using a mirrored digital representation of the intact body part and the digital representation of the impaired body part. Because body parts of limbs occur in pairs and their mirror images are almost identical (chirality), according to the invention the mirrored shape of the intact body part is used for shaping the prosthesis of the impaired body part. There is no need for complex steps for shaping and adapting the prosthesis from a blank. Moreover, a custom prosthesis is produced that mimics the patient's own body, which can provide psychological support for the patient, in particular in the case of recent (partial) amputation of the body part.

In the simplest case, the mirrored digital representation of the intact body part can be reduced by the shape of the digital representation of the impaired body part such that the remaining part constitutes a representation of the prosthesis for the impaired body part. This is preferably carried out by way of 3D processing software, for example a slicer program, such as Geomagic®, Freeform®, or Blender. Further esthetic and/or functional adaptation steps are likewise preferably carried out using 3D processing software. For example, the digital representation of the impaired body part is scaled down slightly to ensure a tight and secure fit of a (subsequent) prosthesis. Furthermore, the transition of the digital representation of the prosthesis to the digital representation of the impaired body part is preferably adapted and/or a wall thickness of the digital representation of the prosthesis is standardized.

In a further method step, at least one prosthesis for the impaired body part is manufactured using the digital representation of the prosthesis and a 3D printer with a print resolution of less than 0.1 mm. A 3D printer with a print resolution of less than 0.09 mm, less than 0.08 mm, less than 0.07 mm, less than 0.06 mm, or less than 0.05 mm is preferably used. The print resolution preferably refers to a direction along the height direction of the layer thickness of the material used. By limiting the print resolution to a range of less than 0.1 mm, the surfaces of the prosthesis are distinctly smoother than in other additive manufacturing processes, such as for example fused deposition modeling (FDM) printers or fused filament fabrication (FFF) printers which for example allow a print resolution of 0.2 mm to 0.15 mm. As a result, a particularly lifelike prosthesis is provided which produces a high level of wearing comfort and improved body feel for the user of the prosthesis. A 3D printer with a print resolution of less than 0.04 mm, less than 0.03 mm, less than 0.025 mm, or less than 0.02 mm is particularly preferably used to enable still smoother prosthesis surfaces.

A lifelike prosthesis for a body part of a limb can be rapidly and straightforwardly manufactured according to the present invention. The availability of a digital representation of the prosthesis means that numerous prostheses can be manufactured or reproduced with little effort and costs remain low in the long term. Even in the event of changes to the impaired body part over the course of time, it is possible in this way to provide a rapid and uncomplicated response to a change in the body part by adapting the digital representation of the prosthesis, for example using a newly created digital representation of the impaired body part. The prostheses can furthermore be adapted or varied in color, for example to make it possible to offer a selection of different colored prostheses. This makes it possible to take account in particular of seasonal changes in the patient's skin tone.

A preferred development provides that the prosthesis for the impaired body part is printed out directly from the digital representation of the prosthesis by the synthetic resin 3D printer. Direct printing of the prosthesis is a simple and rapid method for physically producing the prosthesis in accordance with its digital representation.

A further preferred development provides that a positive form of the prosthesis and a positive form of the impaired body part are produced using the digital representations of the prosthesis and the impaired body part and using the synthetic resin 3D printer. A negative impression of the prosthesis is furthermore produced using the positive form of the prosthesis and the prosthesis for the impaired body part is manufactured using the negative impression of the prosthesis. In other words, the prosthesis for the impaired body part is in this case printed out indirectly from the digital representation of the prosthesis by the synthetic resin 3D printer. Creating a negative impression of the prosthesis makes it possible to increase the variety of colors and to reduce the costs for manufacturing the prosthesis or numerous prostheses since the manufactured prosthesis is not dependent on the colors specified by the synthetic resins for the 3D printer. As a result, materials that are less costly than the synthetic resin can be used for manufacturing the prosthesis. Silicone rubber is preferably used as the material. Silicone rubbers have particularly good skin compatibility and are easy to process and inexpensive. Silicone is preferably poured into the negative impression of the prosthesis and the positive form of the impaired body part is inserted or pressed into the negative impression of the prosthesis in order to shape the precisely fitting transition of the prosthesis to the impaired body part in the negative impression. The negative impression can be used repeatedly such that here too it is straightforward to manufacture numerous prostheses. Coverage of numerous different skin tones can similarly be provided by appropriate coloration of the silicones. It is similarly possible to provide a response to changes to the impaired body part by updating the digital representation of the impaired body part and printing it out as a new positive form of the impaired body part using the 3D printer. The prosthesis can be provided with an adapted new transition to the impaired body part by pressing the new positive form into the silicone present in the negative impression.

Cold- or room temperature-vulcanizing (RTV) and/or high-temperature-vulcanizing (HTV) silicone rubbers and/or liquid silicone rubbers (LSR) are preferably used. The Shore hardness of the cured silicones is preferably A 30 to A 80, preferably A 30 to A 60, particularly preferably A 50, measured to DIN ISO 48-4. Examples of RTV silicones are REPISIL 35 NO A/B with a Shore hardness of A 35 (to DIN ISO 868) and SF 45-RTV 2 with a Shore hardness of A 45 (to DIN ISO 48-4). An example of a liquid silicone is ELASTOSIL® KR 5040/50 A/B with a Shore hardness of A 50 (to DIN ISO 48-4).

During manufacture of the prosthesis for the impaired body part, a textile material is preferably embedded in the manufactured prosthesis. The embedded textile material serves to reinforce and increase the tear strength of the silicone. This also makes it possible to reduce the wall thickness of the silicone, so saving on material costs and the weight of the prosthesis. A textile material stocking is preferably clamped in the negative impression of the prosthesis and the silicone poured into the negative impression of the prosthesis. Pressing in the positive form of the impaired body part ensures that the silicone envelops the stocking. A nylon stocking is preferably used. Nylon is particularly tear-resistant.

Vent holes and/or mold locks are also preferably introduced into the digital representations of the impaired body part and of the prosthesis. The vent holes (also denoted vent channels) enable better venting of the silicone in the negative impression of the prosthesis. Providing mold locks ensures that the positive form of the impaired body part is correctly located in the negative impression of the prosthesis, so simplifying manufacture. The mold locks are preferably configured such that there is only one possible arrangement.

A further preferred development provides that the prosthesis for the impaired body part comprises a pressure relief pad made of a soft silicone and/or of a soft 3D-printed synthetic resin. The digital representation of the impaired body part (20) is preferably lengthened by an intended thickness of the pressure relief pad and/or preferably a pressure relief region intended for the pressure relief pad is added to the digital representation of the prosthesis (22). The pressure relief pad acts as a buffer between the prosthesis and the body part, so improving body feel and wearing comfort for the patient. In other words, insertion of the pressure relief pad into the prosthesis to be manufactured is planned before it is manufactured by appropriately processing the digital representation of the prosthesis for the impaired body part or the digital representation of the impaired body part. This hugely simplifies the manufacture of a prosthesis with a pressure relief pad. The prosthesis for the impaired body part and the pressure relief pad are preferably formed in one piece, so improving the longevity and durability of the prosthesis. The term “one piece” means that no further material, i.e., a binder, adhesive, fastening element, or the like, is required for (subsequently) connecting the prosthesis to the pressure relief pad. The soft silicone and/or the soft 3D-printed synthetic resin preferably has/have a Shore hardness of 00-10 to A 30, preferably 00-20 to A 5, particularly preferably 00-30 to 00-50 or 00-20 to 00-30, measured to DIN ISO 48-4. Examples of soft silicones are the addition-vulcanizing silicones of the ECOFLEX® range. A soft 3D-printed synthetic resin is obtained, for example, by reducing the intended material density of the synthetic resin used in the pressure relief region. For example, the number (and thus the density) of the 3D printing points can be reduced and/or inclusions, i.e., cavities, can be provided in the 3D-printed synthetic resin in the pressure relief region. Additionally or alternatively, a further (different) 3D-printable synthetic resin can be used to form the pressure relief pad in the pressure relief region, wherein the further synthetic resin is softer than the synthetic resin used for the rest of the prosthesis.

The digital representation of the impaired body part is preferably lengthened by an intended thickness of the pressure relief pad. During manufacture of the prosthesis for the impaired body part using the negative impression of the prosthesis, a first silicone is then poured into the negative impression, the positive form of the lengthened impaired body part is pressed into the negative impression and, once the first silicone has cured, a second silicone is poured onto the first silicone in the negative impression. The second silicone is softer than the first silicone. This enables simple manufacture of a prosthesis with a one-piece pressure relief pad. The one-piece structure is obtained by the second silicone adhering to the first cured silicone during vulcanization (in the course of the process). The first silicone is preferably the above-stated silicone, while the second silicone is preferably the silicone described as soft. The prosthesis for the impaired body part is preferably printed out from the digital representation of the prosthesis by the 3D printer and the softness of the 3D-printed synthetic resin in the pressure relief region is achieved by reducing the material density of the synthetic resin and/or by using a further 3D-printed synthetic resin, wherein the further synthetic resin is softer than the synthetic resin used for the rest of the prosthesis. The reduction in material density causes the printed-out synthetic resin to become more elastic and thus softer. The reduced material density is preferably introduced into the digital representation of the prosthesis prior to 3D printing, such that it is obtained directly on printing. This enables simple manufacture of a 3D-printed prosthesis with a one-piece pressure relief pad. The one-piece structure is obtained by the further synthetic resin being printed onto the synthetic resin used for the rest of the prosthesis during 3D printing and so adhering to the rest of the synthetic resin (in the course of the process).

A further preferred development provides that the limb's color shade is determined and the prosthesis is manufactured in a color on the basis of the determined color shade. A color scanner system is preferably used to determine skin tone. Examples are portable color sensors from Spectromatch or Nix Pro. Reference photos of the limbs additionally or alternatively can be used. Using the determined color shade of the limb, it is possible to manufacture a prosthesis that is as similar as possible to the patient's skin and so obtain a lifelike rendering. This can improve patient acceptance and thus the wearing comfort of the prosthesis. An external appearance of the intact body part is preferably imitated by applying color to the prosthesis. This can be done using an airbrush gun, brush, or the like.

A further preferred development provides that the 3D printer used is an SLS or synthetic resin 3D printer. Selective laser sintering (SLS) 3D printers are distinguished by high resolution and by layer transitions that are substantially invisible (to the human eye), i.e., no individual visible layers. When an SLS 3D printer is used, a layer of finely divided powder is introduced into the printer and fused by a laser to form a part. Synthetic resin 3D printers are distinguished by their particular print quality. Synthetic resin 3D printers use 3D printing methods such as stereolithography (SLA) or digital light processing (DLP), as a result of which resolution is determined by the optical dot of the laser or projector (SLA) or of the pixel (DLP). Since light is used for polymerization, no force is applied during printing, which means that the surfaces are distinctly smoother than with other additive manufacturing methods, such as for example with fused deposition modeling (FDM) printers or fused filament fabrication (FFF) printers. In particular, layer thicknesses, i.e., a print resolution, of below 0.08 mm, for example of 0.04 mm to 0.08 mm, can be achieved with SLS 3D printers, and layer thicknesses, i.e., a print resolution, of below 0.025 mm, for example between 0.025 mm and 0.01 mm, can be achieved with synthetic resin 3D printers. A further embodiment preferably uses a 3D printer based on dual-color photoinitiators. In this method, a material containing a dual-color photoinitiator is illuminated from different directions with light of different wavelengths corresponding to the absorption colors of the photoinitiators and, where the light of the different wavelengths impinges on a dual-color photoinitiator, selective materialization occurs such that a 3D model to be produced can be produced by sectional images obtained from the model.

A further preferred development provides that the size of the digital representation of the prosthesis and/or of the digital representation of the impaired body part is adapted such that the manufactured prosthesis can be attached to the impaired body part by way of a suction effect. For example, the size of one of the digital representations is adapted by 90 per cent to 110 per cent, preferably by 92 per cent to 108 per cent. In particular in the case of smaller limbs, such as fingers, sufficient grip can be ensured in this way, such that no additional attaching means are required. For larger limbs, attachment devices known in principle to the skilled person, such as tabs, latching elements, and the like, can be provided.

A further preferred development provides that a negative impression of the impaired body part and a negative impression of the associated intact body part are produced. A positive form of the impaired body part and a positive form of the intact body part are furthermore produced using the negative impressions. In this case, the digital representations of the impaired body part and of the intact body part are created using the positive forms. By using negative impressions, it is possible to transfer fine surface structures onto the positive forms. In this case, all that is required is to create the negative impressions of the patient which then allow digital representations to be produced regardless of location. For example, the negative impressions of the patient are created and sent to another location to be used there in accordance with the invention. Consequently, the patient need not be present for the creation of the digital representations. In addition, there is no blurring caused by patient movement during creation of the digital representations. The negative impressions are preferably manufactured using silicone, alginate, or gypsum. The positive forms are preferably cast with gypsum, polyurethanes (PU), polymer gypsum, or a similar material using the negative impressions.

A further preferred development provides that the manufactured prosthesis comprises a core made of a silicone blend with fillers. Using a core with fillers enables 25 per cent to 33 per cent weight savings in the prosthesis. The wall thickness of the outer layer of the prosthesis is then preferably between 0.5 cm and 1 cm. Cores made of silicone blends with fillers can particularly advantageously be used for foot prostheses, in particular forefoot prostheses. In this way, it is possible, for example, to make a weight saving of the order of magnitude of 400 g in the case of an adult forefoot prosthesis.

A further preferred development provides that a recessed region for an object to be inserted is provided in the digital representation of the prosthesis. For example, a fingernail or toenail is removed from the digital representation so that there is a corresponding space in the manufactured prosthesis for attachment of an artificial fingernail or toenail. Acrylic nails are preferably used. Attaching objects, such as toenails or fingernails, makes the prosthesis even more lifelike, so increasing wearing comfort. Additionally or alternatively, esthetic blemishes can also be removed.

A further aspect relates to device for manufacturing a prosthesis for a body part of a limb. The device comprises an acquisition unit that is configured to create digital representations of impaired and intact body parts of a limb. The acquisition unit is preferably a 3D scanner that is configured to create high-density meshes. The device furthermore comprises an electronic control unit that is configured to create a digital representation of a prosthesis for the impaired body part using a mirrored digital representation of the intact body part and the digital representation of the impaired body part, and a synthetic resin 3D printer that is configured to manufacture a prosthesis for the impaired body part using the digital representation of the prosthesis and/or positive forms of the digital representations of the prosthesis and of the impaired body part using the digital representations of the prosthesis and of the impaired body part. The optional features described in relation to the method and the advantages thereof can be implemented mutatis mutandis with the device and can therefore be combined with one another as desired.

A preferred development provides that the device furthermore comprises a molding kit for making negative impressions.

The above-stated control unit is preferably implemented by electrical or electronic parts or components (hardware) or by firmware (ASIC). Additionally or alternatively, the functionality of the control unit is implemented by executing a suitable program (software). The control unit is likewise preferably implemented by a combination of hardware, firmware, and/or software. For example, individual components of the control unit for providing individual functionalities are embodied as a separate integrated circuit or arranged on a common integrated circuit.

The individual components of the control unit are furthermore preferably embodied as one or more processes that run on one or more processors in one or more electronic computing devices and are produced on execution of one or more computer programs. The instructions of the computer programs are preferably stored in a memory, such as for example a RAM element. The computer programs can, however, also be stored in a nonvolatile storage medium, such as for example a CD-ROM, a flash memory, or the like.

It is furthermore apparent to a person skilled in the art that it is possible to combine the functionalities of a plurality of computing units (data processing devices) or to combine them in a single device or that the functionality of a given data processing device can be distributed over a plurality of devices in order to implement the functionality of the control unit.

A further aspect relates to a computer program comprising commands that, on execution of the program by a computer, such as for example a device for manufacturing a prosthesis for a body part of a limb, cause said computer to carry out the method according to the invention, in particular a method for manufacturing a prosthesis for a body part of a limb.

Further preferred developments of the invention are apparent from the other features stated in the subclaims.

The various embodiments of the invention set out in this application can advantageously be combined with one another unless otherwise specified.

The invention is described below with reference to exemplary embodiments and on the basis of the associated drawings, in which:

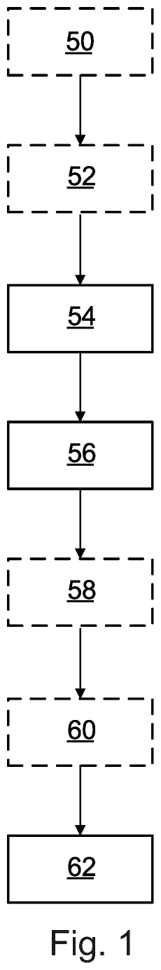

FIG. 1 is a schematic depiction of a method according to one embodiment;

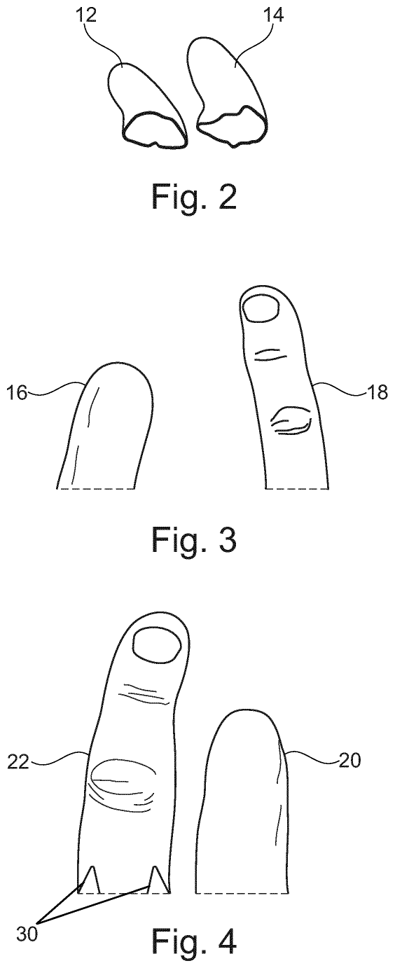

FIG. 2 is a schematic depiction of a negative impression of an impaired body part and a negative impression of an intact body part;

FIG. 3 is a schematic depiction of a positive form of an impaired body part and a positive form of the associated intact body part;

FIG. 4 is a schematic depiction of a digital representation of the impaired body part and a digital representation of the prosthesis;

FIG. 5 is a schematic depiction of a positive form of the prosthesis and a positive form of the impaired body part;

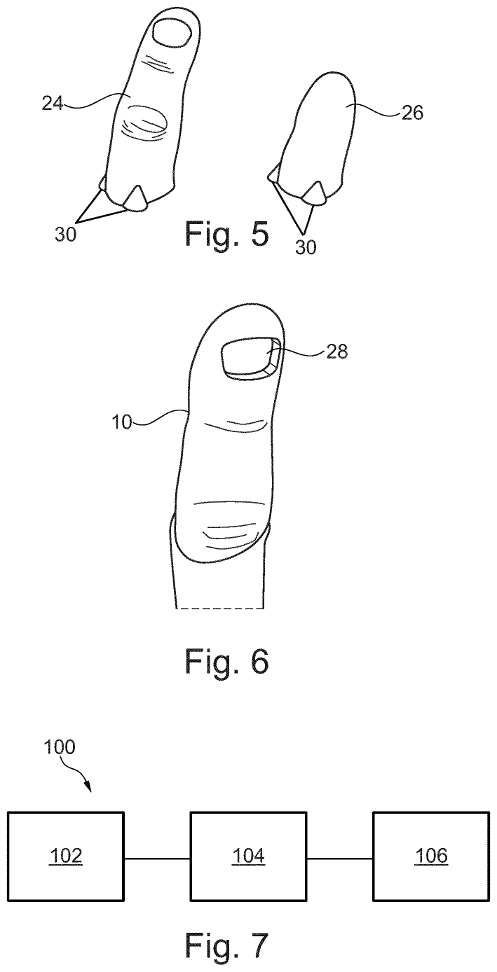

FIG. 6 is a schematic depiction of a prosthesis for a body part of a limb; and

FIG. 7 is a schematic depiction of a device according to one embodiment.

FIG. 1 is a schematic depiction of a procedure for manufacturing a prosthesis 10 for a body part of a limb according to one embodiment. The method shown in FIG. 1 is explained in greater detail with regard to FIGS. 2 to 6 on the basis of a pair of fingers as body parts of a limb, without being limited thereto. The intention is instead merely to illustrate the underlying inventive concept. The method makes it possible to manufacture custom prostheses 10 simply and inexpensively.

In a first method step 50, a negative impression of an impaired body part 12 and a negative impression of the associated intact body part 14 are produced (see FIG. 2). To this end, silicone, alginate, or gypsum is applied to the patient's finger stump and to the associated opposite intact finger and the negative impressions 12, 14 are manufactured. The negative impressions 12, 14 reproduce the fine surface structures of the patient's two fingers. Once the negative impressions 12, 14 have been created, the patient's presence is no longer required, such that manufacture of the prosthesis 10 can continue without the patient. Extremely little effort is thus required on the part of the patient. The negative impressions 12, 14 taken from the patient can, for example, be sent to a specialized laboratory for further processing, for example across borders.

In a second method step 52, a positive form of the impaired body part 16 and a positive form of the intact body part 18 are produced using the negative impressions 12, 14 (see FIG. 3). To this end, gypsum, polyurethanes (PU), or polymer gypsum are pressed into the negative impressions 12, 14 so that the fine surface structures are transferred onto the positive forms 16, 18. The information about individual fingernails, the fingertip and knuckle shapes, and the characteristic wrinkles and creases in the skin of the patient's finger are thus retained. Since the positive forms 16, 18 serve as a physical copy of the patient's pair of fingers, it is important to preserve the characteristic form of the finger and the fine surface structures in order to be able to produce a prosthesis 10 that is as lifelike as possible.

A digital representation of an impaired body part 20 of a limb (see FIG. 4) and a digital representation of an associated intact body part of the limb (not shown) are then created (third method step 54). The digital representations 20 are created using the positive forms 16, 18. High-density meshes of the two fingers are created using a 3D scanner or an imaging method. The digital representations 20 are created in such detail that the characteristic form and fine surface structures of the fingers are included. In this way, structures as small as 0.01 mm can be distinguished from one another, i.e., resolved.

In a fourth method step 56, a digital representation of a prosthesis 22 for the impaired body part is created using a mirrored digital representation of the intact body part and the digital representation of the impaired body part 20 (see FIG. 4 again). The invention makes use of the fact that fingers occur in pairs in nature and the mirror images of pairs of fingers are virtually identical (chirality). A prosthesis for a finger stump, as selected in the present example, can thus be manufactured on the basis of the mirrored shape of the intact finger. Because the digital representation of the intact finger is available, it is child's play to mirror it digitally. As a result, a custom prosthesis that imitates the patient's own body is simply and inexpensively designed. Imitation of the patient's own body can provide psychological support, especially in the case of a recent amputation or recent loss of the finger. Because the intact finger is digitally represented in such accurate detail, the effect can be enhanced by a prosthesis 10 that is manufactured with the same level of detail.

Since the digital representations of the two fingers are available in high resolution, the digital representation of the prosthesis 22 can also easily be designed on a computer using a 3D processing program. To this end, the mirrored digital representation of the intact finger is placed over the digital representation of the finger stump 20 and the overlapping part is removed from the mirrored digital representation of the intact finger. The remaining part of the digital representation of the intact finger now forms the basis for the digital representation of the prosthesis 22 for the finger stump. In order to ensure a better fit, the digital representation of the finger stump 20 is slightly scaled down. Furthermore, a recessed region 28 is inserted into the finger as required (see FIG. 6) by exposing the finger's nail bed in the digital representation of the prosthesis 22 so that an acrylic artificial nail can be adhesively bonded thereon in the subsequent prosthesis 10. The artificial nail makes the prosthesis even more lifelike.

In a fifth method step 58, a positive form of the prosthesis 24 and a positive form of the impaired body part 26 are produced using the digital representations of the prosthesis 22 and of the impaired body part 20 (see FIG. 5). A 3D printer with a print resolution of less than 0.1 mm, such as a synthetic resin 3D printer, is used for this purpose. Alternatively, an SLS 3D printer can also be used. It is important for the printer used to be capable of providing the necessary print resolution. It is thus immaterial which 3D printer technology is ultimately used. It is only the print quality of the printer used, such as an SLS or synthetic resin 3D printer or a 3D printer with a comparable resolution with which layer thicknesses of between 0.1 and 0.01 mm are achievable, that makes it possible to transfer the fine surface structures of the digital representations 20, 22 to the physical positive forms.

In a sixth method step 60, a negative impression of the prosthesis is created on the basis of the printed-out positive form of the prosthesis 24. The created negative impression of the prosthesis is now available for repeated use in order to produce a plurality of prostheses 10, for example also from different materials and/or color blends. In comparison with directly printing the prosthesis 10 using the 3D printer, the selection of material and color is not limited to the materials specified by the 3D printer, such as finely divided powders or synthetic resins. As a result, less costly materials, such as silicone rubber, can be used to manufacture the prosthesis 10. Silicone rubbers are additionally skin-compatible and readily processable.

In a seventh method step 62, at least one prosthesis 10 is manufactured using the negative impression of the prosthesis and the positive form of the impaired body part 26. To this end, silicone to be crosslinked is poured into the negative impression of the prosthesis and the positive form of the impaired body part 26 is pressed into the negative impression of the prosthesis in order to shape the precisely fitting transition of the prosthesis 10 to the finger stump. Once the silicone has vulcanized, the prosthesis 10 retains the shape predetermined by the negative impression as well as the transition predetermined by the positive form of the impaired body part 26. Since the negative impression can be used repeatedly, a plurality of prostheses 10 for different skin tones can be manufactured using appropriately colored silicones. This accordingly provides the patient with a large selection of different colored prostheses that can be inexpensively and quickly produced.

It is also straightforward to respond to changes in the finger stump by creating a new digital representation of the finger stump 20 and creating a new positive form of the finger stump 26 using the 3D printer. In most cases, there is no need to modify, i.e., to reprint, the positive form of the prosthesis 24 such that the existing negative impression of the prosthesis can be reused. A prosthesis 10 with an adapted new transition to the changed finger stump can be manufactured by pressing the new positive form of the finger stump 26 into the silicone present in the negative impression.

As furthermore shown in FIG. 4, mold locks 30 can be inserted into the digital representations of the impaired body part 20 (not shown) and the prosthesis 22. Providing mold locks 30 ensures that the positive form of impaired body part 26 (see FIG. 5) is correctly located in the negative impression of the prosthesis, so simplifying manufacture. The mold locks 30 are preferably configured such that there is only one possible arrangement of the positive form of the impaired body part 26 in the negative impression of the prosthesis. The mold locks 30 are, for example, conical in shape.

It should be borne in mind in the above description that method steps 50, 52, 58 and 60 are in principle of an optional nature for the invention. This fact is indicated in FIG. 1 by method steps 50, 52, 58 and 60 being depicted in dashed boxes. For example, the digital representations of the pair of fingers can alternatively be produced directly on the patient's pair of fingers using a 3D scanner. It is moreover possible for the prosthesis 10 to be printed out directly in accordance with the digital representation of the prosthesis 22.

Finally, FIG. 7 is another schematic depiction of a device 100 according to one embodiment. The device 100 is suitable for manufacturing a prosthesis 10 for a body part of a limb, in particular for manufacturing a prosthesis 10 for a finger stump described with regard to the method according to FIG. 1 and in FIGS. 2 to 6.

The device 100 comprises an acquisition unit 102, an electronic control unit 104, and a 3D printer 106 with a print resolution of less than 0.1 mm. The 3D printer 106 is preferably an SLS or synthetic resin 3D printer. The acquisition unit 102 is configured to create digital representations of impaired 20 and intact body parts of a limb. The electronic control unit 104 is configured to create a digital representation of a prosthesis 22 for the impaired body part using a mirrored digital representation of the intact body part and a digital representation of the impaired body part 20. The 3D printer 106 is set up to manufacture at least one prosthesis 10 for the impaired body part using the digital representation of the prosthesis 22. This can be carried out either directly or indirectly, for example making use of a molding kit for making negative impressions (not shown).

### LIST OF REFERENCE NUMERALS

10 Prosthesis for a body part of a limb
12 Negative impression of an impaired body part
14 Negative impression of the associated intact body part
16 Positive form of an impaired body part
18 Positive form of the associated intact body part
20 Digital representation of the impaired body part
22 Digital representation of the prosthesis
24 Positive form of the prosthesis
26 Positive form of the impaired body part
28 Recessed region
30 Mold locks
50 First method step
52 Second method step
54 Third method step
56 Fourth method step
58 Fifth method step
60 Sixth method step
62 Seventh method step
100 Device for manufacturing a prosthesis for a body part of a limb
102 Acquisition unit
104 Electronic control unit
106 3D printer

1. A method for manufacturing a prosthesis for a body part of a limb, comprising the steps of
creating a digital representation of an impaired body part of a limb and a digital representation of an associated intact body part of the limb;
creating a digital representation of a prosthesis for the impaired body part using a mirrored digital representation of the intact body part and the digital representation of the impaired body part; and
manufacturing at least one prosthesis for the impaired body part using the digital representation of the prosthesis and a 3D printer with a print resolution of less than 0.1 mm.
wherein the prosthesis for the impaired body part comprises a pressure relief pad made of a soft silicone and/or of a soft 3D-printed synthetic resin, wherein the digital representation of the impaired body part is lengthened by an intended thickness of the pressure relief pad and/or a pressure relief region intended for the pressure relief pad is added to the digital representation of the prosthesis.

2. The method according to claim 1, wherein the prosthesis for the impaired body part is printed out directly from the digital representation of the prosthesis by the 3D printer.

3. The method according to claim 1, furthermore comprising the steps of
producing a positive form of the prosthesis and a positive form of the impaired body part using the digital representations of the prosthesis and of the impaired body part and the 3D printer,
producing a negative impression of the prosthesis using the positive form of the prosthesis, and
manufacturing the prosthesis for the impaired body part using the negative impression of the prosthesis.

4. The method according to claim 3, wherein, during manufacture of the prosthesis for the impaired body part, a textile material is embedded in the manufactured prosthesis.

5. The method according to claim 3, wherein vent holes and/or mold locks are introduced into the digital representations of the impaired body part and of the prosthesis.

6. (canceled)

7. The method according to claim 1, wherein, during manufacture of the prosthesis for the impaired body part using the negative impression of the prosthesis, a first silicone is poured into the negative impression, the positive form of the lengthened impaired body part is pressed into the negative impression and, once the first silicone has cured, a second silicone is poured onto the first silicone in the negative impression, wherein the second silicone is softer than the first silicone or wherein the prosthesis for the impaired body part is printed out from the digital representation of the prosthesis by the 3D printer and the softness of the 3D-printed synthetic resin in the pressure relief region is achieved by reducing the material density of the synthetic resin and/or by using a further 3D-printed synthetic resin, wherein the further synthetic resin is softer than the synthetic resin used for the rest of the prosthesis.

8. The method according to claim 1, wherein the limb's color shade is determined and the prosthesis is manufactured in a color on the basis of the determined color shade.

9. The method according to claim 1, wherein the 3D printer used is an SLS or synthetic resin 3D printer.

10. The method according to claim 1, wherein the size of the digital representation of the prosthesis and/or of the digital representation of the impaired body part is adapted such that the manufactured prosthesis can be attached to the impaired body part by way of a suction effect.

11. The method according to claim 1, furthermore comprising the steps of
producing a negative impression of the impaired body part and a negative impression of the associated intact body part and
producing a positive form of the impaired body part and a positive form of the intact body part using the negative impressions, wherein the digital representations of the impaired body part and of the intact body part are created using the positive forms.

12. The method according to claim 1, wherein the manufactured prosthesis comprises a core of a silicone blend with fillers.

13. The method according to claim 1, wherein a recessed region for an object to be inserted is provided in the digital representation of the prosthesis.

14. A device for manufacturing a prosthesis for a body part of a limb, comprising:
an acquisition unit that is configured to create digital representations of impaired and intact body parts of a limb.
an electronic control unit that is configured to:
create a digital representation of a prosthesis for the impaired body part using a mirrored digital representation of the intact body part and a digital representation of the impaired body part; and
lengthen the digital representation of the impaired body part by an intended thickness of a pressure relief pad and/or
add a pressure relief region intended for the pressure relief pad to the digital representation of the prosthesis; and
a 3D printer with a print resolution of less than 0.1 mm that is configured to manufacture at least one prosthesis for the impaired body part using the digital representation of the prosthesis, wherein the prosthesis for the impaired body part comprises the pressure relief pad made of a soft silicone and/or of a soft 3D-printed synthetic resin.

15. The device according to claim 14, furthermore comprising a molding kit for making negative impressions.
